(19) United States
(12) Patent Application Publication
(10) Pub. No.: US 2006/0246099 A1
(43) Pub. Date: Nov. 2, 2006

(54) Compositions containing piperazine compounds

(75) Inventor: Alexandre Cavezza, Pavillon Sous Bois (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/401,914

(22) Filed: Apr. 12, 2006

Related U.S. Application Data
(60) Provisional application No. 60/675,897, filed on Apr. 29, 2005.

(30) Foreign Application Priority Data
Apr. 29, 2005 (FR) 05 51025

(51) Int. Cl. A61K 31/495; A61K 8/49; C07D 241/04

(52) U.S. Cl. 424/401; 514/252.12; 544/399

(57) ABSTRACT

The present invention relates to a composition containing at least one piperazine compound of formula: 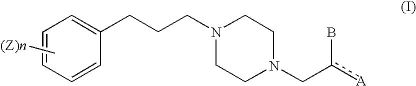 and to a process for treating wrinkled skin, etc. Two novel families of piperazine compounds are also described.

### REFERENCE TO PRIOR APPLICATIONS

This application claims priority to U.S. provisional application 60/675,897 filed Apr. 29, 2005, and to French patent application 0551025 filed Apr. 21, 2005, both incorporated herein by reference.

### FIELD OF THE INVENTION

The present invention relates to a composition comprising a physiologically acceptable medium suitable for topical application to the skin and at least one piperazine compound of given formula. The invention also relates to a process for treating wrinkled skin, comprising the topical application of this composition to the skin, and also to two novel families of piperazine compounds.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Women, and even men, currently have a tendency to wish to look youthful for as long as possible and consequently seek to fade out the age marks on the skin, which are reflected in particular by wrinkles and fine lines. In this respect, advertising and the fashion world report about products intended to keep the skin radiant and wrinkle-free for as long as possible, which are signs of youthful skin, all the more so since the physical appearance acts on the psyche and/or on the morale.

Hitherto, wrinkles and fine lines were treated using cosmetic products containing active agents acting on the skin, for example by improving its cell renewal or alternatively by promoting the synthesis, or by preventing the degradation, of the elastic fibres thereof of which skin tissue is composed.

Although these treatments make it possible to act on the wrinkles and fine lines caused by chronological or intrinsic ageing, and also on those caused by photoageing, they have no effect on expression wrinkles, which require an intervention on the muscular contractile component (via muscle-relaxing agents) or dermal contractile component (via dermo-decontracting agents) of wrinkles.

Specifically, expression wrinkles are the result of mechanisms different from those that generate the wrinkles caused by ageing.

Specifically, they are produced due to the effect of the strain exerted on the skin by the skin muscles that allow facial expressions. Depending on the shape of the face, the frequency of facial expressions and possible tics, they may appear even from childhood. Age, and also certain environmental factors such as exposure to sunlight, do not play a part in generating them, but may make them deeper and permanent.

Expression wrinkles are characterized by the presence of grooves around the orifices formed by the nose (nasal grooves), the mouth (perioral wrinkles and “sour-face” wrinkles) and the eyes (crow's-feet wrinkles), around which are the skin muscles, and also between the eyebrows (glabella wrinkles or lion wrinkles) and on the forehead.

Hitherto, the only means commonly used for acting on expression wrinkles is botulinum toxin, which is especially injected into the wrinkles of the glabella which are wrinkles between the eyebrows (see J. D. Carruters et al., J. Dermatol. Surg. Oncol., 1992, 18, pp. 17-21).

The Assignee company has also proposed various compounds capable of affording a muscle-relaxing effect when they are applied topically to the skin, thus making it possible to act on expression wrinkles via another route. Among these compounds that may especially be mentioned are antagonists of the receptors associated with the calcium channels, such as verapamil (FR-2 793 681), and in particular manganese and its salts (FR-2 809 005) and alverine (FR-2 798 590); and agonists of the receptors associated with the chlorine channels, including glycine (EP-0 704 210) and certain extracts of Iris pallida (FR-2 746 641); and sapogenins (EP-1 352 643).

Along with these muscle-relaxing agents, the Assignee has described various dermo-decontracting compounds and in particular adenosine (EP-1 424 064) and amine compounds (EP-1 405 633).

However, there is still a need for other effective compounds for smoothing or fading out wrinkles, in particular expression wrinkles.

### SUMMARY OF THE INVENTION

One aim of the present invention is to provide compounds with muscle-relaxing properties that at the same time offer improved bioavailability compared with other dermo-decontracting agents or muscle relaxants such as the known calcium inhibitors.

The inventor has now discovered, surprisingly, that certain piperazine compounds satisfy this need.

### DETAILED DESCRIPTION OF THE PREFERRED EMBODIMENTS

One subject of the present invention is thus a composition comprising a physiologically acceptable medium suitable for topical application to the skin and at least one piperazine compound chosen from compounds of formula (I): 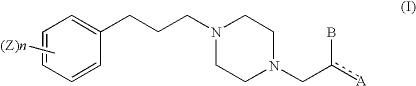
in which:
each of the groups Z independently denotes a fluorine atom, a linear C₁-C₆ or branched C₃-C₆ alkyl radical, a linear C₂-C₆ or branched C₃-C₆ alkenyl radical, a C₃-C₆ cycloalkyl radical, a CF₃ group, a group OR₁, a group NR₁R₂, a group COR₁, a group COOR₁, a group CONR₁R₂ or a group N(R₁)COR₂;
n ranges from 0 to 5;
A denotes:
a hydrogen atom,
a linear C₁-C₂₀ or branched C₃-C₂₀ alkyl radical,
a linear C₂-C₂₀ or branched C₃-C₂₀ alkenyl radical,
a C₃-C₂₀ cycloalkyl radical,
   the radicals being optionally substituted with at least one group chosen from: an oxo radical, a group OR₁, a group NR₁R₂, a group COR₁, a group COOR₁, a group CONR₁R₂, a group N(R₁)COR₂ or a phenyl radical optionally substituted with one to five groups Z as defined above,
a phenyl radical optionally substituted with one to five groups Z as defined above;
B denotes a hydrogen atom, a linear C₁-C₆ or branched C₃-C₆ alkyl radical, a linear C₂-C₆ or branched C₃-C₆ alkenyl radical, a C₃-C₆ cycloalkyl radical, a group OR₁, a group NR₁R₂, a group COR₁, a group COOR₁, a group CONR₁R₂ or a group N(R₁) COR₂;
   in which R₁ and R₂ independently denote a hydrogen atom, a linear C₁-C₆ or branched C₃-C₆ alkyl radical optionally substituted with a phenyl radical, a linear C₂-C₆ or branched C₃-C₆ alkenyl radical, a C₃-C₆ cycloalkyl radical or a phenyl radical; the groups R₁, or, respectively, R₂, possibly being identical or different;
   and in which the dashed lines denote a potential double bond, it being understood that A is other than, e.g., a hydrogen atom or a phenyl radical when this double bond is present,
   and the salts, optical isomers and solvates thereof.

A subject of the invention is also the cosmetic use of the abovementioned composition for skincare, in particular for caring for wrinkled skin and/or for smoothing out wrinkles, relaxing lines and/or decontracting the skin.

A subject of the invention is also the cosmetic use of at least one piperazine compound as defined above, as an agent for attenuating wrinkles and/or decontracting the skin and/or relaxing lines.

A subject of the invention is also a cosmetic process for treating wrinkled skin, in particular the skin of the face and/or the forehead, comprising the topical application to the skin of a composition as defined above.

For the purposes of the present invention, the term “wrinkles” in particular means the wrinkles on the face, including the forehead, and on the neck. These are more particularly expression wrinkles caused by repeated facial expressions and accentuated with age. The wrinkles concerned are preferably those lying radially around the mouth and/or the eyes, in particular crow's-feet wrinkles, and/or located on the forehead, in particular the “lion” wrinkle in the glabella, the space between the eyebrows, and/or lying horizontally on the forehead.

The composition according to the invention is advantageously intended to be applied to the areas of the face and/or the forehead marked with expression wrinkles, and/or to individuals with expression wrinkles.

Moreover, the term “physiologically acceptable medium” means a medium that is compatible with the skin and more particularly with the skin of the face and/or the neck. This medium is advantageously cosmetically acceptable, i.e. it does not cause any unacceptable itching, stinging or redness liable to put the user off the composition, and it has a pleasant appearance, odour and feel.

In formula (I), the alkyl groups may especially be chosen, depending on the case, from the following groups: methyl, ethyl, n-propyl, isopropyl, n-butyl, isobutyl, tert-butyl, pentyl, hexyl, heptyl, octyl, nonyl, decyl, undecyl, dodecyl, myristyl and palmityl.

In addition, in the context of the present patent application, the term “alkenyl” means radicals possibly comprising one or more conjugated or non-conjugated double bonds. They may especially be chosen, depending on the case, from the following groups: vinyl, allyl, butenyl and pentenyl. When A in formula 1 is alkenyl and the double bond of Formula I represented as a dashed line is present, the alkenyl unsaturation is preferably not terminal unsaturation oriented towards the ring—i.e, a 1,2-diene (allene) structure ═C═ is preferably avoided.

Finally, examples of cycloalkyl radicals are cyclobutyl, cyclopentyl and cyclohexyl radicals.

The term “substituted” refers to radicals, in particular alkyl radicals, which may be substituted with side groups and/or with groups located at the end of the chain of the radicals. Thus, the term “alkyl radical substituted with a phenyl radical” especially means radicals otherwise known as “aralkyl”.

As salts of the compound of formula (I), mention may be made of the salts obtained by adding the compound of formula (I) to a mineral acid, chosen especially from hydrochloric acid, sulfuric acid and phosphoric acid, or to an organic acid, chosen in particular from acetic acid, propionic acid, succinic acid, fumaric acid, lactic acid, glycolic acid, citric acid and tartaric acid.

Preferably, the piperazine compound according to the invention is such that when B is a hydrogen atom, then A is other than an optionally substituted phenyl group.

In addition, according to one advantageous embodiment of the invention, each of the groups Z independently denotes a group chosen from: a fluorine atom, a group OR₁, a group NR₁R₂, a group N(R₁) COR₂, a group COOR₁ or a CF₃ group. More preferentially, Z denotes a group OR₁ or a group COOR₁, preferably a group OR₁. According to one preferred embodiment, R₁ and R₂ thus independently denote: a hydrogen atom or a linear C₁-C₆ or branched C₃-C₆ alkyl radical, preferably a methyl or ethyl radical.

As a variant, the piperazine compound according to the invention is such that n=0.

In addition, according to one advantageous embodiment of the invention, A denotes:
a hydrogen atom,
a linear C₁-C₈ or branched C₃-C₈ alkyl radical,
a linear C₂-C₈ or branched C₃-C₈ alkenyl radical,
a C₃-C₈ cycloalkyl radical,
   the radicals being optionally substituted with at least one group chosen from: an oxo radical, a group OR₁, a group NR₁R₂ or a phenyl radical optionally substituted with one to five groups Z as defined in formula (I), or
a phenyl radical optionally substituted with one to five groups Z as defined in formula (I).

Better still, it is preferable for A to denote a hydrogen atom or a linear C₁-C₆ or branched C₃-C₆ alkyl radical optionally substituted with at least one group chosen from: an oxo radical, a group OR₁, or a phenyl radical, in which R₁ preferably denotes a hydrogen atom and may denote a linear C₁-C₆ or branched C₃-C₆ alkyl radical such as a methyl radical.

Moreover, the preferred piperazine compounds according to the invention are such that B denotes a hydrogen atom; a linear C₁-C₆ or branched C₃-C₆ alkyl radical; a group NR₁R₂; a group COR₁; or a group OR₁, in which R₁ denotes a hydrogen atom or a linear C₁-C₆ or branched C₃-C₆ alkyl radical optionally substituted with a phenyl radical.

Better still, B denotes a hydrogen atom, a group OR₁ or a group COR₁ in which R₁ denotes a linear C₁-C₆ or branched C₃-C₆ alkyl radical optionally substituted with a phenyl radical.

Among the compounds of formula (I) described above, the ones preferably used are those for which:
A denotes a linear C₁-C₈ alkyl radical optionally substituted with a phenyl radical, B denotes a hydrogen atom or a hydroxyl group, and n=0; or
A denotes a hydrogen atom, B denotes —OR₁ or —COR₁ with R₁ denoting a linear C₁-C₆ alkyl radical optionally substituted with a phenyl radical, and n=0.

Preferentially, the compounds of formula (I) that are used are those for which:
A denotes a linear C₁-C₈ alkyl radical optionally substituted with a phenyl radical, B denotes a hydrogen atom and n=0; or
A denotes a hydrogen atom, B denotes —OR₁ with R₁ denoting a linear C₁-C₆ alkyl radical substituted with a phenyl radical, and n=0; or
A denotes a C₁-C₈ alkyl radical, B denotes —OH and n=0; or
A denotes a hydrogen atom, B denotes —COR₁ with R₁ denoting a linear C₁-C₆ alkyl radical, and n=0.

Some piperazine compounds that are preferred for use in the present invention are listed in Table 1 below. This table also indicates the molecular weight and cLogP values of the compounds according to the invention. The cLogP value may be calculated as described in the Advanced Chemistry Development software (ACD/Labs) Software Solaris V4.67.

In regards to the bioavailability of cosmetic compounds, it has been demonstrated that in order to cross the stratum corneum, which is the surface layer of the skin, cosmetic compounds and in particular ionizable compounds such as piperazines need to have a molecular weight of less than 400 and preferably a molecular weight of less than 350, and/or a cLogP value (calculated octanol/water partition coefficient) preferably of less than 5 and if possible less than 4.

As emerges from Table 1, the compounds according to the invention all have a molecular weight of less than 400 and/or a cLogP value of less than 5.

For comparative purposes, cinnarizine and flunarizine, which are two reference anti-calcium agents with a structure similar to that of the compounds according to the invention, have a cLogP value of 6.0 and 6.3, respectively.

Thus, the compounds according to the invention have better bioavailability than the known anti-calcium agents of similar structure.

TABLE 1
Examples of piperazine compounds according to the invention

| Ex. | RN | NAME | cLogP | MW |
|---|---|---|---|---|
| 1 | 048203-82-3 | 1-(3-phenyl-2-propenyl)-4- | 4.5 | 320 |
| | | (3-phenyl-propyl)piperazine | | |
| 2 | 056233-37-5 | 1-(2-phenylethyl)-4- | 4.0 | 308 |
| | | (3-phenylpropyl)-piperazine | | |
| 3 | 067469-80-1 | 1-(2-hydroxyethyl)-4- | 1.7 | 248 |
| | | (3-phenylpropyl)-piperazine | | |
| 4 | 067514-13-0 | 1-(3-hydroxypropyl)-4- | 1.6 | 262 |
| | | (3-phenylpropyl)-piperazine | | |
| 5 | 076087-26-8 | 1-ethyl-4-(3- | 2.5 | 232 |
| | | phenylpropyl)piperazine | | |
| 6 | 076087-28-0 | 1-propyl-4-(3- | 3.1 | 246 |
| | | phenylpropyl)piperazine | | |
| 7 | 076087-32-6 | 1-butyl-4-(3- | 3.6 | 260 |
| | | phenylpropyl)piperazine | | |
| 8 | 076087-34-8 | 1-(2-methylpropyl)-4-(3- | 3.4 | 260 |
| | | phenylpropyl)-piperazine | | |
| 9 | 076087-36-0 | 1-pentyl-4-(3- | 4.1 | 274 |
| | | phenylpropyl)piperazine | | |
| 10 | 076087-38-2 | 1-hexyl-4-(3- | 4.7 | 288 |
| | | phenylpropyl)piperazine | | |
| 11 | 076087-40-6 | 1-heptyl-4-(3- | 5.2 | 302 |
| | | phenylpropyl)piperazine | | |
| 12 | 076087-44-0 | 1-(3-phenylpropyl)-4-(2- | 2.9 | 244 |
| | | propenyl)piperazine | | |
| 13 | 076087-46-2 | 1-(2-butenyl)-4-(3- | 3.4 | 258 |
| | | phenylpropyl)piperazine | | |
| 14 | 076087-54-2 | 1,4-bis(3- | 4.6 | 322 |
| | | phenylpropyl)piperazine | | |
| 15 | 076087-68-8 | 1-[N,N-diethyl(2-aminoethyl)]-4- | 3.2 | 303 |
| | | (3-phenylpropyl)piperazine | | |
| 16 | 082075-48-7 | 1-(3-phenylpropan-3-one)-4- | 3.7 | 336 |
| | | (3-phenylpropyl)piperazine | | |
| 17 | 086621-39-8 | ethyl 4-[2-[4-(3-phenylpropyl)- | 4.5 | 380 |
| | | 1-piperazinyl]ethyl]benzoate | | |
| 18 | 086621-53-6 | 4-[2-[4-(3-phenylpropyl)1- | 3.6 | 352 |
| | | piperazinyl]ethyl]benzoic acid | | |
| 19 | 165377-43-5 | 1-[2-(3,4-dimethoxyphenyl)ethyl]- | 3.7 | 368 |
| | | 4-(3-phenylpropyl)piperazine | | |
| 20 | 197451-51-7 | 1-[2-(3,4- | 3.8 | 386 |
| | | dimethoxyphenyl)ethyl]-4-[3-(4- | | |
| | | fluorophenyl)propyl]piperazine | | |
| 21 | 200885-91-2 | 1-(2-phenylpropyl)-4-(3- | 4.3 | 322 |
| | | phenylpropyl)piperazine | | |
| 22 | 219659-23-1 | 2-[2-[4-(3-phenylpropyl)-1- | 2.7 | 323 |
| | | piperazinyl]-ethyl]benzenamine | | |
| 23 | 219659-24-2 | 3-[2-[4-(3-phenylpropyl)-1- | 2.7 | 323 |
| | | piperazinyl]-ethyl]benzenamine | | |
| 24 | 219659-25-3 | 4-[2-[4-(3-phenylpropyl)-1- | 2.7 | 323 |
| | | piperazinyl]-ethyl]benzenamine | | |
| 25 | 219659-26-4 | 1-[2-(2-methoxyphenyl)ethyl]- | 3.9 | 338 |
| | | 4-(3-phenylpropyl)piperazine | | |
| 26 | 219659-27-5 | 1-[2-(3-methoxyphenyl)ethyl]- | 3.9 | 338 |
| | | 4-(3-phenyl-propyl)piperazine | | |
| 27 | 219659-28-6 | 1-[2-(4-methoxyphenyl)ethyl]- | 3.9 | 338 |
| | | 4-(3-phenylpropyl)piperazine | | |
| 28 | 219659-40-2 | 1-(2-aminoethyl)-4-(3- | 1.3 | 247 |
| | | phenylpropyl)piperazine | | |
| 29 | 219659-41-3 | 1-(3-aminopropyl)-4-(3- | 1.7 | 261 |
| | | phenylpropyl)-piperazine | | |
| 30 | 219659-42-4 | 1-(4-aminobutyl)-4-(3- | 2.0 | 275 |
| | | phenylpropyl)piperazine | | |
| 31 | 422575-11-9 | N-methyl-N-(2-phenylethyl)-1- | 4.5 | 380 |
| | | (3-aminopropyl-4-(3- | | |
| | | phenylpropyl)piperazine | | |
| 32 | 425644-73-1 | 2-methoxy-4-[2-[4- | 2.9 | 354 |
| | | (3-phenylpropyl)-1- | | |
| | | piperazinyl]ethyl]phenol | | |
| 33 | 652159-28-9 | 2-methoxy-4-[2-[4- | 3.0 | 372 |
| | | [3-(4-fluorophenyl)propyl]- | | |
| | | 1-piperazinyl]ethyl]phenol | | |
| 34 | 652159-29-0 | 1-[2-(4-ethoxy-3- | 4.2 | 382 |
| | | methoxyphenyl)ethyl]-4- | | |
| | | (3-phenylpropyl)piperazine | | |
| 35 | 652159-30-3 | 1-[2-(4-ethoxy-3- | 4.3 | 400 |
| | | methoxyphenyl)ethyl]-4-[3-(4- | | |
| | | fluorophenyl)propyl]piperazine | | |
| 36 | 690200-18-1 | 1-[4-(4-fluorophenyl)butan-4- | 4.1 | 398 |
| | | one]-4-[3-(4- | | |
| | | methoxyphenyl)propyl]piperazine | | |
| 37 | 703397-45-9 | 1-(3-hydroxy-2-phenylpropyl)- | 3.3 | 338 |
| | | 4-(3-phenylpropyl)piperazine | | |
| 38 | 712261-46-6 | 1-[2-(2,5- | 3.9 | 369 |
| | | dimethoxyphenyl)ethyl]- | | |
| | | 4-(3-phenylpropyl)piperazine | | |
| 39 | 732964-55-5 | 1-[3-(3,4- | 4.8 | 397 |
| | | dimethoxyphenyl)propyl]-4- | | |
| | | (4-phenylbutyl)piperazine | | |
| 40 | 745775-91-1 | 4-[3-[4-[2-(3,4- | 3.0 | 384 |
| | | dimethoxyphenyl)ethyl]-1- | | |
| | | piperazinyl]propyl]phenol | | |
| 41 | 759433-88-0 | 1-[3-(3,4- | 4.4 | 382 |
| | | dimethoxyphenyl)propyl]- | | |
| | | 4-(3-phenylpropyl)piperazine | | |
| 42 | novel | 1-[4-(3-phenylpropyl)piperazin- | 4.2 | 330 |
| | | 1-yl]-octan-3-one | | |
| 43 | novel | 1-[4-(3-phenylpropyl)piperazin- | 3.8 | 332 |
| | | 1-yl]-octan-3-ol | | |
| 44 | novel | 1-[2-(benzyloxy)-ethyl]- | 3.8 | 338 |
| | | 4-(3-phenylpropoyl)piperazine | | |

The piperazine compounds according to the invention may especially be prepared according to the following reaction scheme: 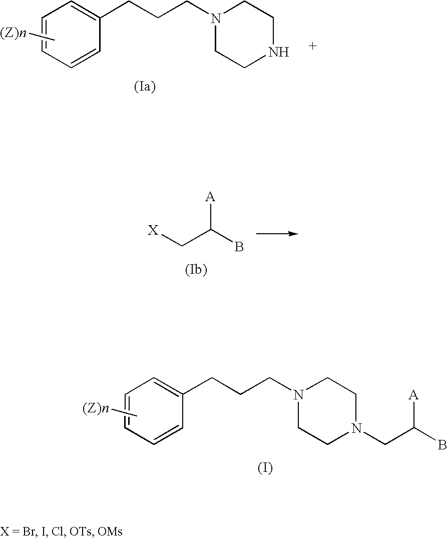
by reacting 1-(3-phenylpropyl)piperazine (Ia) (1 eq.) predissolved in a polar organic solvent (0.5 to 1M) such as methanol, ethanol, acetonitrile, DMF or DMSO, optionally supplemented with a mineral base (1 to 5 eq.) such as NaHCO₃, Na₂CO₃, NaOH or KOH, with reagent (Ib) (1 eq.). The mixture is stirred for one to ten hours, optionally with heating to reflux. The reaction is then worked up and the product is purified via standard methods.

Reagent (Ia) may be prepared by reacting 1 equivalent of piperazine with 1 equivalent of 1-chloro-3-arylpropyl in a polar solvent such as dimethylformamide, according to the process described in J. Med. Chem. 1998, 25, 4950.

As a variant, reagent (Ib) may be replaced with reagent (Ic):

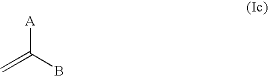

The piperazine compounds according to the invention may also be prepared as described by Zikolova, S. Bul. Trudova 1978, 10, 33-46 or by Matecka, D. et al. J. Med. Chem. 1996, 32(24), 4704-4716.

Certain piperazine compounds are described in the prior art, especially in the following documents:

Compounds 2 and 14 are described in patent application WO 2004/092 123.

Compound 3 is described in documents FR-A-2 374 318 and U.S. Pat. No. 5,177,077.

Compound 4 is described in patent application FR-A-2 374 322.

Compound 5 is described in patent application JP-A-2005-239 594.

Compounds 17 and 18 are described in patent U.S. Pat. No. 4,616,086.

Compound 19 is described in documents WO 2001/00604, WO 2002/030 422, U.S. Pat. No. 6,492,369 and U.S. Pat. No. 5,736,546.

Compound 21 is described in the article Rische et al., “One-pot synthesis of secondary and tertiary amines by carbonylative hydroaminomethylation of alkenes catalysed by di(μ-chloro)bis(η4-1,5-cyclooctadiene)-dirhodium”, Synthesis (1997), (11), 1331-1337.

Compounds 22, 23, 27 and 28 are described in patent application US 2003-171 347.

Compounds 22 to 30 are described in the article Zhang et al., “Characterization of novel N,N′-disubstituted piperazines as sigma receptor ligands”, Journal of medicinal chemistry (1998), 41(25), 4950-4957.

Compound 31 is described in the article Benedetti et al., “GBR compounds and mepyramines as cocaine abuse therapeutics: chemometric studies on selectivity using grid independent descriptors”, Journal of medicinal chemistry (2002), 45(8), 1577-1584.

Compounds 20 and 32 to 35 are described in the article Kawamura et al., “Synthesis and evaluation of 11C- and 18F-labeled 1-[2-(4-alkoxy-3-methoxyphenyl)ethyl]-4-(3-phenylpropyl)piperazines as sigma receptor ligands for positron emission tomography studies”, Nuclear medicine and biology (2003), 30(3), 273-284.

Some of the piperazine compounds according to the invention are novel.

A subject of the invention is thus also novel piperazine compounds of formula (IIa) or (IIb): 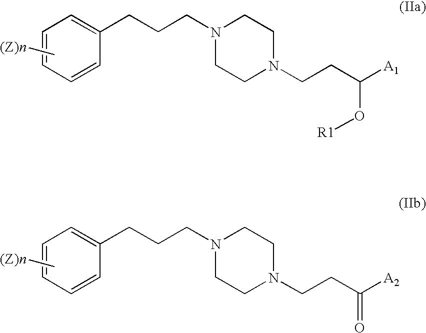
in which:

each of the groups Z independently denotes a fluorine atom, a linear C₁-C₆ or branched C₃-C₆ alkyl radical, a linear C₂-C₆ or branched C₃-C₆ alkenyl radical, a C₃-C₆ cycloalkyl radical, a CF₃ group, a group OR₁, a group NR₁R₂, a group COR₁, a group COOR₁, a group CONR₁R₂ or a group N(R₁)COR₂;

n ranges from 0 to 5;

R₁ and R₂ independently denote a hydrogen atom, a linear C₁-C₆ or branched C₃-C₆ alkyl radical optionally substituted with a phenyl radical, a linear C₂-C₆ or branched C₃-C₆ alkenyl radical, a C₃-C₆ cycloalkyl radical or a phenyl radical; the groups R₁, or, respectively, R₂, possibly being identical or different; and

A₁ and A₂ independently denote a linear C₁-C₁₉ or branched C₃-C₁₉ alkyl radical, a linear C₂-C₁₉ or branched C₃-C₁₉ alkenyl radical or a C₃-C₁₉ cycloalkyl radical, the radicals being optionally substituted with at least one group chosen from: an oxo radical, a group OR₁, a group NR₁R₂, a group COR₁, a group COOR₁, a group CONR₁R₂, a group N(R₁)COR₂ or a phenyl radical optionally substituted with one to five groups Z as defined above.

Preferably, n is equal to 0.

In addition, it is preferable for R₁ to denote a hydrogen atom or a linear C₁-C₆ or branched C₃-C₆ alkyl radical, more preferentially a hydrogen atom.

In addition, A₁ and A₂ advantageously denote a linear C₁-C₆ or branched C₃-C₆ alkyl radical that is preferably not substituted.

Preferably, for the compounds of formula (IIa):

A₁ denotes a linear C₁-C₆ alkyl radical, R₁ denotes a hydrogen atom and n=0, or

A₁ denotes a hydrogen atom, R₁ denotes a linear C₁-C₆ alkyl radical optionally substituted with a phenyl radical, and n=0.

Preferably, for the compounds of formula (IIb), A₂ denotes a linear C₁-C₆ alkyl radical and n=0.

The amount of piperazine compound that may be used according to the invention obviously depends on the desired effect and may thus vary within a wide range.

To give an order of magnitude, these compounds may be used in an amount representing from 0.01% to 10% of the total weight of the composition, preferably in an amount representing from 0.05% to 5% of the total weight of the composition and more preferably in an amount representing from 0.1% to 2% of the total weight of the composition.

This composition may be in any presentation form, including any form normally used in cosmetics, and it may especially be in the form of an optionally gelled solution, a dispersion of the lotion type, optionally a two-phase lotion, an emulsion obtained by dispersing a fatty phase in an aqueous phase (O/W emulsion) or conversely (W/O emulsion), or a triple emulsion (W/O/W or O/W/O emulsion) or a vesicular dispersion of ionic and/or nonionic type. These compositions are prepared according to the usual methods. A composition in the form of an oil-in-water emulsion is preferably used according to this invention.

This composition may be more or less fluid and may have the appearance of a white or coloured cream, an ointment, a milk, a lotion, a serum, a paste or a mousse. It may optionally be applied in the form of an aerosol. It may also be in solid form, in particular in the form of a stick. It may be used as a care product and/or as a makeup product for the skin.

The composition according to the invention may also contain adjuvants such as those that are common in cosmetics, such as hydrophilic or lipophilic gelling agents, hydrophilic or lipophilic active agents, preserving agents, antioxidants, solvents, fragrances, fillers, screening agents, pigments, odour absorbers and dyestuffs. The amounts of these various adjuvants are those conventionally used in the field under consideration, and, for example, from 0.01% to 20% relative to the total weight of the composition. Depending on their nature, these adjuvants may be introduced into the fatty phase, into the aqueous phase, or into lipid vesicles. In any case, these adjuvants, and also the proportions thereof, will be preferably be chosen so as not to harm the desired properties of the compounds according to the invention.

When the composition used according to the invention is an emulsion, the proportion of the fatty phase may range, for example, from 5% to 80% by weight and preferably from 5% to 50% by weight relative to the total weight of the composition. The oils, emulsifiers and co-emulsifiers used in the composition in emulsion form are chosen from those conventionally used in the field under consideration. The emulsifier and co-emulsifier are present in the composition in a proportion ranging from 0.3% to 30% by weight and preferably from 0.5% to 20% by weight relative to the total weight of the composition.

As oils that may be used in the invention, mention may be made of mineral oils (liquid petroleum jelly), oils of plant origin (avocado oil or soybean oil), oils of animal origin (lanolin), synthetic oils (perhydrosqualene), silicone oils (cyclomethicone) and fluoro oils (perfluoropolyethers). Fatty alcohols (cetyl alcohol), fatty acids and waxes (carnauba wax or ozokerite) may also be used as fatty substances.

As examples of emulsifiers and co-emulsifiers that may be used in the invention, mention may be made of fatty acid esters of polyethylene glycol such as PEG-100 stearate, and fatty acid esters of glycerol such as glyceryl stearate.

Hydrophilic gelling agents/thickeners that may be mentioned in particular include carboxyvinyl polymers (carbomer), acrylic copolymers such as acrylate/alkylacrylate copolymers, polyacrylamides, polysaccharides, natural gums and clays, and lipophilic gelling agents/thickeners that may be mentioned include modified clays, for instance bentones, metal salts of fatty acids and hydrophobic silica.

As active agents, it will be advantageous to introduce into the composition used according to the invention at least one compound chosen from: desquamating agents; moisturizers; depigmenting or propigmenting agents; antiglycation agents; NO-synthase inhibitors; agents for stimulating the synthesis of dermal or epidermal macromolecules and/or for preventing their degradation; agents for stimulating the proliferation of fibroblasts and/or keratinocytes or for stimulating the differentiation of keratinocytes; other muscle-relaxing agents and/or dermo-decontracting agents; tensioning agents; antipollution agents and/or free-radical scavengers; agents acting on the capillary circulation; agents acting on the energy metabolism of cells; and mixtures thereof.

Examples of such additional compounds are: retinol and its compounds such as retinyl palmitate; ascorbic acid and its compounds such as magnesium ascorbyl phosphate and ascorbyl glucoside; tocopherol and its compounds such as tocopheryl acetate; nicotinic acid and its precursors such as nicotinamide; ubiquinone; glutathione and its precursors such as L-2-oxothiazolidine-4-carboxylic acid; plant extracts and especially plant proteins and hydrolysates thereof, and also plant hormones; marine extracts such as algal extracts; bacterial extracts; sapogenins such as diosgenin and extracts of Dioscorea plants and especially of wild yam, containing diosgenin; ceramides; hydroxy acids such as salicylic acid and 5-n-octanoylsalicylic acid; resveratrol; oligopeptides and pseudodipeptides and acyl compounds thereof; manganese and magnesium salts, in particular the gluconates; and mixtures thereof.

As mentioned previously, the composition according to the invention may also contain UVA-active and/or UVB-active photoprotective agents, in the form of organic or mineral compounds, the latter optionally being coated to make them hydrophobic.

The organic photoprotective agents may be chosen especially from: anthranilates, in particular menthyl anthranilate; benzophenones, in particular benzophenone-1, benzophenone-3, benzophenone-5, benzophenone-6, benzophenone-8, benzophenone-9, benzophenone-12 and, preferably, benzophenone-3 (oxybenzone) or benzophenone-4 (Uvinul MS40 available from BASF); benzylidenecamphors, in particular 3-benzylidenecamphor, benzylidenecamphorsulfonic acid, camphor benzalkonium methosulfate, polyacrylamidomethylbenzylidenecamphor, terephthalylidenedicamphorsulfonic acid and, preferably, 4-methylbenzylidenecamphor (Eusolex 6300 available from Merck); benzimidazoles, in particular benzimidazilate (Neo Heliopan AP available from Haarmann & Reimer), or phenylbenzimidazolesulfonic acid (Eusolex 232 available from Merck); benzotriazoles, in particular drometrizole trisiloxane, or methylenebis-benzotriazolyltetramethylbutylphenol (Tinosorb M available from Ciba); cinnamates, in particular Cinoxate, DEA methoxycinnamate, diisopropyl methylcinnamate, glyceryl ethylhexanoate dimethoxycinnamate, isopropyl methoxycinnamate, isoamyl cinnamate and, preferably, ethocrylene (Uvinul N35 available from BASF), octyl methoxycinnamate (Parsol MCX available from Hoffmann La Roche), or octocrylene (Uvinul 539 available from BASF); dibenzoylmethanes, in particular butylmethoxydibenzoylmethane (Parsol 1789); imidazolines, in particular ethylhexyl dimethoxybenzylidene dioxoimidazoline; PABAs, in particular ethyl dihydroxypropyl PABA, ethylhexyldimethyl PABA, glyceryl PABA, PABA, PEG-25 PABA and, preferably, diethylhexylbutamidotriazone (Uvasorb HEB available from 3V Sigma), ethylhexyltriazone (Uvinul T150 available from BASF) or ethyl PABA (benzocaine); salicylates, in particular dipropylene glycol salicylate, ethylhexyl salicylate, homosalate or TEA salicylate; triazines, in particular anisotriazine (Tinosorb S available from Ciba); drometrizole trisiloxane.

The mineral photoprotective agents preferably consist of zinc oxide and/or titanium dioxide, preferably of nanometric size, optionally coated with alumina and/or stearic acid.

The invention will now be illustrated by means of the non-limiting examples that follow. In these examples, the amounts are indicated as weight percentages.

### EXAMPLES

### Example 1

### Synthesis of 1,4-bis(3-phenylpropyl)piperazine

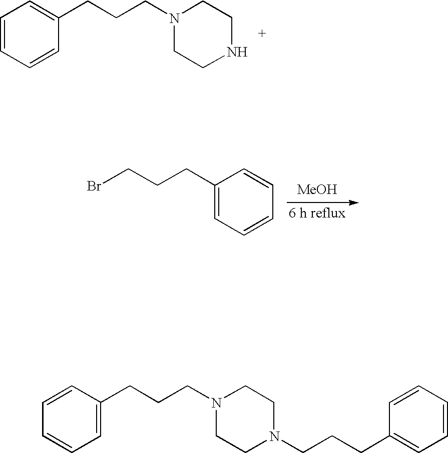

1 g (4.9 mmol) of 1-(3-phenylpropyl)piperazine was dissolved in 20 ml of methanol in a round-bottomed flask. 0.85 ml (5.4 mmol) of 1-bromo-3-phenylpropane was then added and the mixture was refluxed for 6 hours. The reaction medium was then cooled to room temperature and diluted with ethyl acetate and water. The aqueous phase was extracted with ethyl acetate. The organic phases were then combined, washed with saturated NaCl solution, dried and concentrated under vacuum. The crude residue was purified on a column of silica (EtOAc/heptane) to give 0.65 g (yield=41%) of 1,4-bis(3-phenylpropyl)piperazine in the form of an oil.

The mass spectrometry and NMR analyses were in accordance with the expected structure.

### Example 2

### Synthesis of 1-[(2E)-3-phenylprop-2-enyl]-4-(3-phenylpropyl)piperazine

This compound may be obtained by following the protocol of Example 2, using cinnamoyl bromide instead of 1-bromo-3-phenylpropane.

This compound is especially sold by the company Chembridge (ref. 5428132).

### Example 3

### Synthesis of 1-[4-(3-phenylpropyl)piperazin-1-yl]octan-3-one

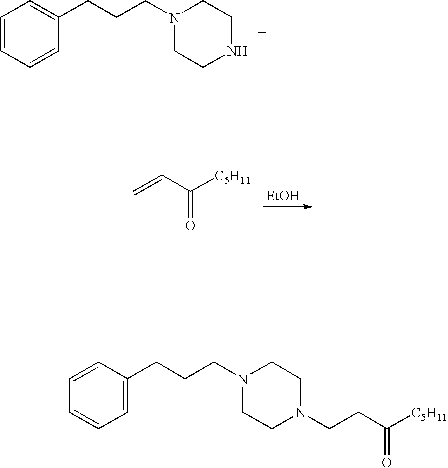

1 g (4.9 mmol) of 1-(3-phenylpropyl)piperazine is dissolved in 10 ml of ethanol in a round-bottomed flask. 0.620 g of 1-octen-3-one (4.9 mmol) is added and the mixture is reacted for 15 hours at room temperature. The reaction medium is diluted with ethyl acetate and water. The aqueous phase is extracted with ethyl acetate. The organic phases are then combined, washed with saturated NaCl solution, dried and concentrated under vacuum. The crude residue is purified on a column of silica (EtOAc/heptane) to give 1.3 g (yield=81%) of 1-[4-(3-phenylpropyl)piperazin-1-yl]octan-3-one in the form of a colourless oil.

The mass spectrometry and NMR analyses are in accordance with the expected structure.

### Example 4

### Synthesis of 1-[4-(3-phenylpropyl)piperazin-1-yl]octan-3-ol

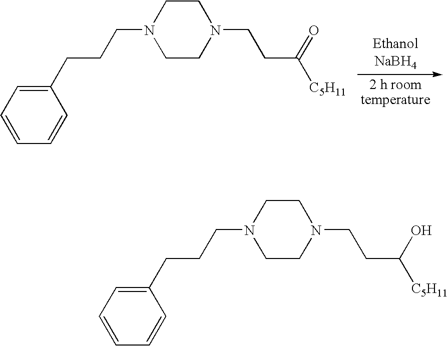

1.62 g (4.9 mmol) of 1-[4-(3-phenylpropyl)piperazin-1-yl]octan-3-one are dissolved in 10 ml of ethanol in a round-bottomed flask. 0.278 g of sodium borohydride granules (7.35 mmol) is added and the mixture is reacted for 2 hours at room temperature. Acetone is then added to remove the excess reducing agent. The reaction medium is diluted with ethyl acetate and water. The aqueous phase is extracted with ethyl acetate. The organic phases are then combined, washed with saturated NaCl solution, dried and concentrated under vacuum. The crude residue is purified on a column of silica (EtOAc/heptane) to give 1.1 g (yield=67%) of 1-[4-(3-phenylpropyl)piperazin-1-yl]octan-3-ol in the form of a colourless oil.

The mass spectrometry and NMR analyses are in accordance with the expected structure.

### Example 5

### Synthesis of 1-[2-(benzyloxy)ethyl]-4-(3-phenylpropyl)piperazine

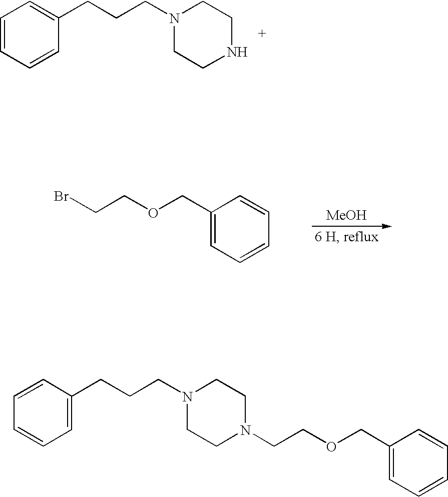

1 g (4.9 mmol) of 1-(3-phenylpropyl)piperazine was dissolved in 20 ml of methanol in a round-bottomed flask. 0.8 ml of benzyl 2-bromoethyl ether (4.9 mmol) was added. The mixture was then refluxed for 6 hours. The reaction medium was cooled to room temperature and diluted with ethyl acetate and water. The aqueous phase was extracted with ethyl acetate. The organic phases were then combined, washed with saturated NaCl solution, dried and concentrated under vacuum. The crude residue was purified on a column of silica (EtOAc/heptane) to give 1.3 g (yield=79%) of 1-[2-(benzyloxy)ethyl]-4-(3-phenylpropyl)piperazine in the form of a yellow oil.

The mass spectrometry and NMR analyses were in accordance with the expected structure.

### Example 6

### Synthesis of 1-heptyl-4-(3-phenylpropyl)-piperazine

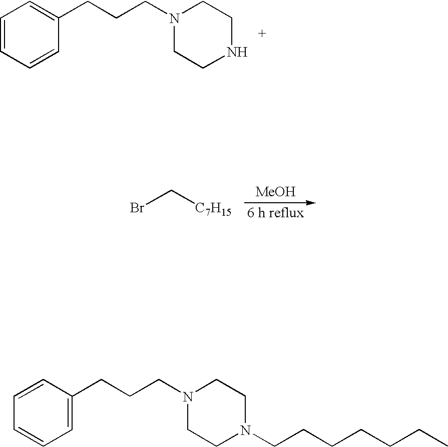

1 g (4.9 mmol) of 1-(3-phenylpropyl)piperazine was dissolved in 25 ml of methanol in a round-bottomed flask. 1.17 ml (4.9 mmol) of 1-bromoheptane were added and the mixture was refluxed for 6 hours. The reaction medium was then cooled to room temperature and diluted with ethyl acetate and water. The aqueous phase was extracted with ethyl acetate. The organic phases were then combined, washed with saturated NaCl solution, dried and concentrated under vacuum. The crude residue was purified on a column of silica (EtOAc/heptane) to give 0.9 g (yield=44%) of 1-heptyl-4-(3-phenylpropyl)piperazine in the form of an oil.

The mass spectrometry and NMR analyses were in accordance with the expected structure.

### Example 7

### Demonstration of the Muscle-Relaxing Effect of the Compounds According to the Invention

### Example 7A

### Activity in Nerve-Muscle Coculture Test

The compounds of Examples 1 and 2 were tested on a model of nerve-muscle coculture, which makes it possible to recreate a motor arc by innervating human striated muscle cells with explants of rat embryonic rachidian ganglia and spinal cord.

This test is predictive of an anti-wrinkle effect, as demonstrated in the case of diazepam, which inhibited muscle fibre contractions in this model and whose anti-wrinkle activity has been demonstrated in vivo.

a) Protocol

Human muscle cells, obtained from samples of striated muscle from healthy donors, are inoculated into wells of cross section 1.8 cm² (24-well culture dishes). After culturing for ten days, these cells form a monolayer and fuse. At this stage, explants of embryonic spinal cord from a 13-day-old rat, containing the rachidian ganglia, are placed on the culture.

The growth of the neurites is visible beyond the spinal cord explant after one day of culture. The first contractions of the muscle fibres are observed after five to six days of coculture, and after three weeks all the muscle fibres in the region of the explants contract.

The cocultures are used after 21 days, when the muscle fibres are striated and contain mature differentiated neuromuscular junctions.

A muscle fibre showing regular contractions (at least 60 contractions per minute) is then selected from three different culture wells and the number of contractions is counted over 30 seconds. The test compounds, diluted in DMSO, are then incubated for 60 seconds in these wells, at a concentration of 10 μM for the compound of Example 1 and of 10 and 100 μM for the compound of Example 2. At the end of incubation, the number of contractions is again counted over 30 seconds. The test is performed in triplicate.

b) Results

| | | % inhibition |
|---|---|---|
| Compound | Concentration | of contractions |
| Example 1 | 10 μM | 65% |
| | 100 μM | 100% |
| Example 2* | 10 μM | 50% |
| | 100 μM | 100% |

*Sold by Chembridge (ref.: 5428132)

The compounds according to the invention thus inhibit striated muscle contraction and may thus be used for relaxing the lines of the face and for smoothing out expression wrinkles.

### Example 7B

### Activity in a Test of Binding to Type L Calcium Channels

a) Protocol

The capacity of the compounds of Examples 1, 4 and 5 (at 1 μM in DMSO) to competitively inhibit the binding of type L calcium-channel agonists was evaluated.

The studies are performed using rat cerebral cortex homogenates (isolated membranes containing at their surface type L calcium channels) according to the method described by Reynolds I. J. et al., 1986, J. Pharmacol. Exp. Ther., 237, p. 731.

The experimental conditions are as follows:

| | Ligand | Non- | | |
|---|---|---|---|---|
| Test | Conc. | specific | Incubation | Detection |
| Ca²⁺ channel | [³H] (−) | D 600 | 60 min/ | scintillation |
| (L, verapamil | 0.5 nM | (10 μM) | 22° C. | counting |
| site) | D 888 | | | |

D888, which is [³H] (−)-demethoxyverapamil, serves as a radiolabelled specific ligand, and D600, which is (±)-methoxyverapamil hydrochloride, serves as a reference molecule.

The specific binding of a ligand (labelled D888) to the receptors (type L calcium channels, verapamil site) is defined as being the difference between the total binding and the non-specific binding determined in the presence of an excess of cold (non-radioactive) ligand. The results are expressed as a percentage of inhibition of the specific binding of the control in the presence of the test compound.

b) Results

| COMPOUND | % inhibition of binding |
|---|---|
| Example 1 | 30% |
| Example 4 | 22% |
| Example 5 | 16% |

The compounds according to the invention thus inhibit the specific binding of the control to the calcium channels.

In addition, a similar test showed that the compound of Example 2 inhibited the specific binding of the control to the calcium channels by 50%, at a concentration of 50 μM.

From this test and from the teaching of patent application EP-1 053 745, it is deduced that these compounds have a high probability of having a beneficial effect on wrinkles and in particular on expression wrinkles.

### Example 8

### Cosmetic Composition

This composition is prepared in a conventional manner for a person skilled in the art. The amounts given in this example are indicated in weight percentages.

| | |
|---|---|
| Compound of Example 2 | 0.10% |
| Stearic acid | 3.00% |
| Mixture of glyceryl monostearate and | 2.50% |
| polyethylene glycol stearate (100 EO) | |
| Polyethylene glycol stearate (20 EO) | 1.00% |
| Cyclopentadimethylsiloxane | 10.00% |
| Fillers | 3.00% |
| Plant oils | 7.00% |
| Synthetic oils | 6.00% |
| Preserving agents | 1.20% |
| Oxyethylenated dimethylsiloxane (16 EO) | 1.00% |
| containing methoxy end groups | |
| Silicone gum | 0.20% |
| Acrylic copolymer as an inverse emulsion | 1.70% |
| (Simulgel 600 from SEPPIC) | |
| Stearyl alcohol | 1.00% |
| Water qs | 100% |

This cream is intended to be applied to the face and the forehead to relax the features and to decontract facial skin.

The above written description of the invention provides a manner and process of making and using it such that any person skilled in this art is enabled to make and use the same, this enablement being provided in particular for the subject matter of the appended claims, which make up a part of the original description and including a composition comprising a physiologically acceptable medium suitable for topical application to the skin, which contains at least one piperazine compound chosen from the compounds of formula (I) described above.

As used above, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The invention method and composition is preferably used by subjects desirous of the benefits noted herein, subjects “in need of” these benefits. Such subjects are typically suffering from, e.g., the appearance of wrinkles and/or lines, such as by self diagnosis or cosmetician or medical diagnosis, or are at recognized and appreciated risk of developing such conditions and who use the invention methods and compositions to combat these effects (e.g., for attenuating wrinkles and/or decontracting the skin and/or relaxing lines). In this regard, the invention process can be viewed as one for treating and/or delaying the onset of the appearance of, and/or for reducing signs of, wrinkles and lines.

Naturally, one using the invention as disclosed will use an amount of the invention composition effective to reduce the signs of wrinkles and lines. Such amount is inclusive of an amount of the compositions described herein at the disclosed concentrations of active ingredients sufficient to cover the area of the skin being treated in a single application, and of course includes that amount applied upon repeated application, for example on a daily basis over a course of days, weeks, etc. In a preferred embodiment the invention process includes multiple applications of the invention composition to the area(s) of skin in need of attention.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in a physiologically acceptable medium suitable for topical application to the skin, at least one piperazine compound chosen from compounds of formula (I): 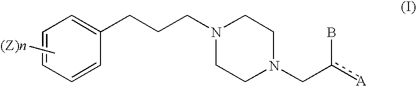
in which:
each of the groups Z independently denotes a fluorine atom, a linear C₁-C₆ or branched C₃-C₆ alkyl radical, a linear C₂-C₆ or branched C₃-C₆ alkenyl radical, a C₃-C₆ cycloalkyl radical, a CF₃ group, a group OR₁, a group NR₁R₂, a group COR₁, a group COOR₁, a group CONR₁R₂ or a group N(R₁)COR₂;
n ranges from 0 to 5;
A denotes:
a hydrogen atom,
a linear C₁-C₂₀ or branched C₃-C₂₀ alkyl radical,
a linear C₂-C₂₀ or branched C₃-C₂₀ alkenyl radical,
a C₃-C₂₀ cycloalkyl radical,
the alkyl, alkenyl and cycloalkyl radicals being optionally substituted with at least one group chosen from: an oxo radical, a group OR₁, a group NR₁R₂, a group COR₁, a group COOR₁, a group CONR₁R₂, a group N(R₁)COR₂ or a phenyl radical optionally substituted with one to five groups Z as defined above,
a phenyl radical optionally substituted with one to five groups Z as defined above;
B denotes a hydrogen atom, a linear C₁-C₆ or branched C₃-C₆ alkyl radical, a linear C₂-C₆ or branched C₃-C₆ alkenyl radical, a C₃-C₆ cycloalkyl radical, a group OR₁, a group NR₁R₂, a group COR₁, a group COOR₁, a group CONR₁R₂ or a group N(R₁)COR₂;
wherein R₁ and R₂ independently denote a hydrogen atom,
a linear C₁-C₆ or branched C₃-C₆ alkyl radical optionally substituted with a phenyl radical, a linear C₂-C₆ or branched C₃-C₆ alkenyl radical, a C₃-C₆ cycloalkyl radical or a phenyl radical; the groups R₁, or, respectively, R₂, optionally being identical or different;
the dashed line denotes a potential double bond, variable A being other than a hydrogen atom or a phenyl radical when this double bond is present,
and the salts, optical isomers and solvates thereof.

2. The composition according to claim 1, wherein the piperazine compound is such that: when B is a hydrogen atom, then A is other than an optionally substituted phenyl group.

3. The composition according to claim 1, wherein each of the groups Z independently denotes a group chosen from: a fluorine atom, a group OR₁, a group NR₁R₂, a group NR₁COR₂, a group COOR₁ and a CF₃ group.

4. The composition according to claim 1, wherein Z denotes a group OR₁ or COOR₁.

5. The composition according to claim 3, wherein R₁ and R₂ independently denote: a hydrogen atom or a linear or branched C₁-C₆ alkyl radical.

6. The composition according to claim 1, wherein n=0.

7. The composition according to claim 1, wherein A denotes:
a hydrogen atom,
a linear C₁-C₈ or branched C₃-C₈ alkyl radical,
a linear C₂-C₈ or branched C₃-C₈ alkenyl radical,
a C₃-C₈ cycloalkyl radical,
the alkyl, alkenyl and cycloalkyl radicals being optionally substituted with at least one group chosen from: an oxo radical, a group OR₁, a group NR₁R₂ or a phenyl radical optionally substituted with one to five groups Z as defined in formula (I), or
a phenyl radical optionally substituted with one to five groups Z as defined in formula (I).

8. The composition according to claim 7, wherein A denotes a hydrogen atom or a linear C₁-C₆ or branched C₃-C₆ alkyl radical optionally substituted with at least one group chosen from: an oxo radical, a group OR₁, or a phenyl radical, in which R₁ denotes a hydrogen atom or a linear C₁-C₆ or branched C₃-C₆ alkyl radical.

9. The composition according to claim 1, wherein B denotes a hydrogen atom; a linear C₁-C₆ or branched C₃-C₆ alkyl radical; a group NR₁R₂; a group COR₁; or a group OR₁, in which R₁ denotes a hydrogen atom or a linear C₁-C₆ or branched C₃-C₆ alkyl radical optionally substituted with a phenyl radical.

10. The composition according to claim 9, wherein B denotes a hydrogen atom, a group OR₁ or a group COR₁ in which R₁ denotes a linear C₁-C₆ or branched C₃-C₆ alkyl radical optionally substituted with a phenyl radical.

11. The composition according to claim 1, wherein:
A denotes a linear C₁-C₈ alkyl radical optionally substituted with a phenyl radical, B denotes a hydrogen atom or a hydroxyl group, and n=0; or
A denotes a hydrogen atom, B denotes —OR₁ or —COR₁ with R₁ denoting a linear C₁-C₆ alkyl radical optionally substituted with a phenyl radical, and n=0.

12. A method for caring for wrinkled skin and/or for smoothing out wrinkles and/or for relaxing lines and/or for decontracting the skin, comprising applying the composition of claim 1 to skin in need thereof.

13. The method of claim 12, wherein said method is a method for caring for wrinkled skin and/or for smoothing out wrinkles.

14. The method of claim 13, comprising the topical application to the skin of the face and/or the forehead of a composition as defined in claim 1.

15. The method of claim 14, wherein the composition is applied to the areas of the face and/or the forehead marked with expression wrinkles.

16. Piperazine compounds of formula (IIa) and (IIb): 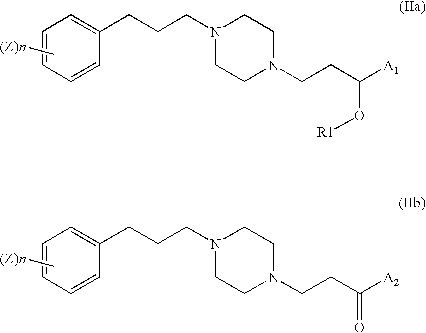
in which:
each of the groups Z independently denotes a fluorine atom, a linear C₁-C₆ or branched C₃-C₆ alkyl radical, a linear C₂-C₆ or branched C₃-C₆ alkenyl radical, a C₃-C₆ cycloalkyl radical, a CF₃ group, a group OR₁, a group NR₁R₂, a group COR₁, a group COOR₁, a group CONR₁R₂ or a group N(R₁)COR₂;
n ranges from 0 to 5;
R₁ and R₂ independently denote a hydrogen atom, a linear C₁-C₆ or branched C₃-C₆ alkyl radical optionally substituted with a phenyl radical, a linear C₂-C₆ or branched C₃-C₆ alkenyl radical, a C₃-C₆ cycloalkyl radical or a phenyl radical; the groups R₁, or, respectively, R₂, possibly being identical or different; and
A₁ and A₂ independently denote a linear C₁-C₁₉ or branched C₃-C₁₉ alkyl radical, a linear C₂-C₁₉ or branched C₃-C₁₉ alkenyl radical or a C₃-C₁₉ cycloalkyl radical, the alkyl and alkenyl radicals being optionally substituted with at least one group chosen from: an oxo radical, a group OR₁, a group NR₁R₂, a group COR₁, a group COOR₁, a group CONR₁R₂, a group N(R₁)COR₂ or a phenyl radical optionally substituted with one to five groups Z as defined above.

17. Piperazine compounds according to claim 16, wherein n is equal to 0.

18. Piperazine compounds according to claim 16, wherein R₁ denotes a hydrogen atom or a linear C₁-C₆ or branched C₃-C₆ alkyl radical.

19. Piperazine compounds according to claim 18, wherein R₁ denotes a hydrogen atom.

20. Piperazine compounds according to claim 16, wherein A₁ and A₂ denote a linear C₁-C₆ or branched C₃-C₆ unsubstituted alkyl radical.

21. Piperazine compounds according to claim 16, wherein said compounds are of formula (IIa) and:
A₁ denotes a linear C₁-C₆ alkyl radical, R₁ denotes a hydrogen atom and n=0 or
A₁ denotes a hydrogen atom, R₁ denotes a linear C₁-C₆ alkyl radical optionally substituted with a phenyl radical, and n=0.

22. Piperazine compounds according to claim 16, wherein said compounds are of formula (IIa) and wherein A₂ denotes a linear C₁-C₆ alkyl radical and n=0.

23. Piperazine compounds according to claim 16, wherein said compounds are of formula (IIa).

24. Piperazine compounds according to claim 16, wherein said compounds are of formula (IIb).
